(19) United States
(12) Patent Application Publication
(10) Pub. No.: US 2009/0305338 A1
(43) Pub. Date: Dec. 10, 2009

(54) Plant cell lines established from the medicinal plant veratrum californicum

(76) Inventors: Anneli Ritala-Nurmi, Helsinki (FI); Heiko Rischer, Espoo (FI); Kirsi-Marja Oksman-Caldentey, Helsinki (FI); Ma Rui, Jilin (CN)

Correspondence Address: TRASKBRITT, P.C., P.O. BOX 2550, SALT LAKE CITY, UT, 84110, US

(21) Appl. No.: 12/310,869

(22) PCT Filed: Sep. 26, 2007
(86) PCT No.: PCT/EP2007/060201

§ 371 Date: Jun. 3, 2009

(30) Foreign Application Priority Data
Sep. 29, 2006 (EP) 06121472.2

(51) Int. Cl. C12P 33/20; C12N 5/04

(52) U.S. Cl. 435/53; 435/410; 435/420; 435/419

(57) ABSTRACT

The present invention relates to the field of plant secondary metabolites. More particularly plant cell lines are established from the medicinal plant Veratrum californicum. Said plant cell lines can be used for the production of cyclopamine and related steroid alkaloids and their precursors.

### FIELD OF THE INVENTION

The present invention relates to the field of plant secondary metabolites. More particularly plant cell lines are established from the medicinal plant Veratrum californicum. Said plant cell lines can be used for the production of cyclopamine and related steroid alkaloids and their precursors.

### BACKGROUND TO THE INVENTION

Plants provide not only foods, but also other useful materials such as wood, cellulose, gums and rubbers. They contain a wide range of chemical compounds including pharmaceuticals, flavours, fragrance, colours and insecticides. These compounds belong to a group collectively known as secondary products or secondary metabolites. The genus Veratrum comprises up to 45 species of perennial herbs distributed throughout the northern temperate to arctic regions of Europe, Asia and North America. Its exact systematic position is still under debate but modern systems place it within the tribe Melanthieae of Melanthiaceae whereas traditionally the genus belongs to the large family of Liliaceae (1). Veratrum is phytochemically characterised by the presence of steroid alkaloids which exhibit interesting pharmacological properties already recognised in ancient times. The teratogenic species Veratrum californicum Durand is distributed throughout the mountains of the western USA and often referred to as the corn lily, was noticed when a high percentage of sheep grazing these areas gave birth to deformed lambs. Only offspring of ewes which had consumed Veratrum during early pregnancy developed the anomalies varying from cyclopia, i.e. extreme craniofacial malformation, to mildly deformed upper jaws (2). Two steroid alkaloids cyclopamine (11-deoxojervine) and jervine have been identified as the responsible teratogens (FIG. 1). More recently the molecular mode of action for cyclopamine-induced teratogenesis has been investigated and it has been revealed that the compound selectively blocks Sonic hedgehog signal transduction (3). This pathway has a central role in a multitude of developmental processes. Interestingly, a number of genes in the Sonic hedgehog signaling network have been associated with certain human tumors. Therefore, cyclopamine and its derivatives are proposed as potential therapeutic agents for the treatment of tumors arising from disruption of components of the hedgehog pathway (4). Cyclopamine has already shown promise in the treatment of medulloblastoma tumor (5), basal cell carcinoma (6) and small cell lung cancer (7) in vitro and in whole animal systems. At present, Veratrum californicum is the only source for cyclopamine because the compound with its complex chemical structure cannot be synthesized at an economical price. Isolation of the substance from wild plants is impeded by the low and highly variable quantity, and is therefore very expensive. In vitro cultures for the production of valuable secondary metabolites have long been recognized as a means of avoiding these shortcomings. Thus it would theoretically be possible to grow large quantities of biomass for the production of complex secondary products by fermentation. But in practice, this is not always the case. This is due to the non-amenability of many plants to grow in culture. Furthermore, of those that could be finally grown under suitable conditions, many lacked desired biosynthetic activity of the commercially most important secondary products. In the present invention we have generated an in vitro culture system for Veratrum californicum comprising the initiation of callus, the establishment of cell suspension cultures, their long-term storage by cryo-preservation, the formation of shoot cultures and the regeneration of plants. In addition, the isolated cell lines surprisingly show the presence of Veratrum steroid alkaloids such as cyclopamine and jervine.

### FIGURES

FIG. 1: Chemical structures of cyclopamine (1) and jervine (2)

FIG. 2: Plant regeneration from embryogenic calli of Veratrum californicum (regeneration medium: L2 without hormone)

Means±standard division are from 5 replicates

|t|=6.130>t_{0.05}=2.306 (P<0.001) for green plant

|t|=0.40<t_{0.05}=2.306 (P>0.05) for albino plant

FIG. 3: Growth of cell suspension line B in medium AA (with 4 mg/l NAA). Fresh weight-closed symbols, dry weight-open symbols. Means±standard division are from 3 replicates.

FIG. 4: Panel A shows the relative cyclopamine contents (as shown in the Y-axis as the response value of detecting the specific cyclopamine ion 124) of individual B cell line samples (n=9, as shown in the X-axis) grown in the light (grey bars) compared to grown in the dark (dark bars). Panel B shows the response values for cyclopamine in the light- (L) and dark- (D) grown B cell line samples. The two groups are significantly different (t-test, p=0.0344).

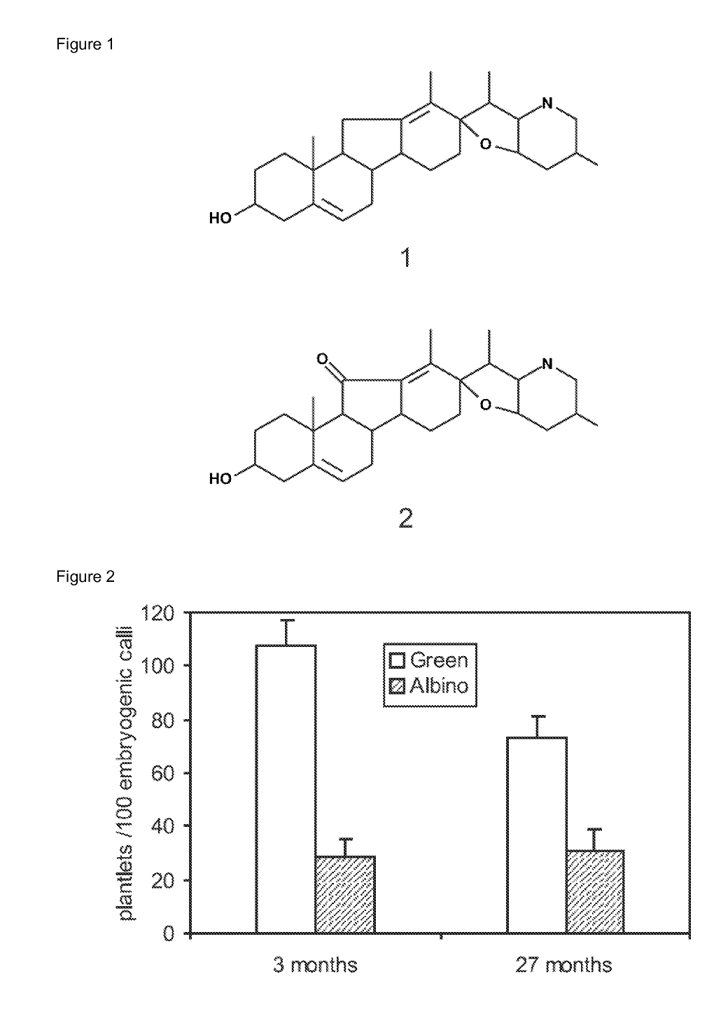

### AIMS AND DETAILED DESCRIPTION OF THE INVENTION

Veratrum californicum (Liliaceae) is an important monocotyledonous medicinal plant which is the only source of the anticancer compound cyclopamine. In the present invention an in vitro culture system for somatic embryogenesis and green plant regeneration of Veratrum californicum was developed. Embryogenic calli were induced from mature embryos on induction medium. Five basal media supplemented with different growth regulators were evaluated for embryogenic callus induction, modified MS medium with 4 mg/l picloram showing the best result for embryogenic callus production. Fine suspension cell lines were established by employing friable embryogenic calli as starting material and AA medium and L2 medium as culture media. The suspension cell lines cultured in M medium with 4 mg/l NM appeared to be fresh yellow and fast growing. The suspension cells were cryopreserved successfully and recovered at a high rate. Green plants were regenerated from embryogenic calli as well as from suspension plant cells. The in vitro plantlets contained the steroid alkaloids cyclopamine and veratramine. These in vitro systems (i.e. callus culture, plant cell line culture and shoot culture) form a springboard for the production of the pharmaceutically important secondary metabolite cyclopamine.

Thus in a first embodiment the invention provides an isolated plant cell line from Veratrum californicum.

In yet another embodiment the isolated plant cell line of Veratrum californicum is capable of producing jervine and cyclopamine. The term ‘capable of’ refers to the fact that said plant cell line produces jervine and cyclopamine. In a particular embodiment said plant cell lines produces jervine and/or cyclopamine upon elicitation with an elicitor. In yet another particular embodiment said elicitor is light, i.e. the plant cell lines are grown in the light.

In yet another embodiment the invention provides a method for the production of a Veratrum californicum plant cell line, said method comprising: a) germinating and isolating mature embryos from Veratrum californicum wherein said embryos are larger than 2 mm, b) cultivating said embryos on modified Murashige and Skoog salts medium supplemented with 4 mg/l-7 mg/l picloram towards embryogenic callus and c) forming a plant suspension culture from said callus in an amino acid based medium supplemented with 4 mg/l 1-naphthaleneacetic acid (NM).

The modified Murashige and Skoog medium (mMS medium) consists of MS basic salts and vitamins, 146 mg/l glutamine and 200 mg/l casein hydrolysate (the composition of MS medium is described in Murashige T and Skoog F (1962) Physiol Plant 15:473-497).

In yet another embodiment said Veratrum calfiornicum plant cell line is obtainable by the production method herein described before.

In yet another particular embodiment said isolated Veratrum californicum plant cell line (either recombinant or not recombinant) is used to produce jervine and/or cyclopamine.

In a particular embodiment said isolated Veratrum californicum plant cell line is a transgenic plant cell line.

A transgenic plant cell line is most commonly generated by using a recombinant DNA vector. The term “recombinant DNA vector” as used herein refers to DNA sequences containing a desired coding sequence and appropriate DNA sequences necessary for the expression of the operably linked coding polynucleotide sequence in the plant cell. Plant cells are known to utilize promoters, polyadenlyation signals and enhancers.

Thus the invention provides a transgenic Veratrum californicum plant or derived cell thereof transformed with said recombinant DNA vector. A recombinant DNA vector comprises at least one “Expression cassette”. Expression cassettes are generally DNA constructs preferably including (5′ to 3′ in the direction of transcription): a promoter region, a polynucleotide sequence, homologue, variant or fragment thereof of the present invention operatively linked with the transcription initiation region, and a termination sequence including a stop signal for RNA polymerase and a polyadenylation signal. It is understood that all of these regions should be capable of operating in biological cells, such as plant cells, to be transformed. The promoter region comprising the transcription initiation region, which preferably includes the RNA polymerase binding site, and the polyadenylation signal may be native to the biological cell to be transformed or may be derived from an alternative source, where the region is functional in the biological cell.

A chosen polynucleotide sequence may be expressed in a plant cell under the control of a promoter that directs constitutive expression or regulated expression. Regulated expression comprises temporally or spatially regulated expression and any other form of inducible or repressible expression. Temporally means that the expression is induced at a certain time point, for instance, when a certain growth rate of the plant cell culture is obtained (e.g. the promoter is induced only in the stationary phase or at a certain stage of development). Spatially means that the promoter is only active in specific organs, tissues, or cells (e.g. only in roots, leaves, epidermis, guard cells or the like). Other examples of regulated expression comprise promoters whose activity is induced or repressed by adding chemical or physical stimuli to the plant cell. In a preferred embodiment the expression is under control of environmental, hormonal, chemical, and/or developmental signals. Such promoters for plant cells include promoters that are regulated by (1) heat, (2) light, (3) hormones, such as abscisic acid and methyl jasmonate (4) wounding or (5) chemicals such as salicylic acid, chitosans or metals. Indeed, it is well known that the expression of secondary metabolites is induced by the addition of for example specific chemicals, jasmonate and elicitors. A constitutive promoter directs expression in a wide range of cells under a wide range of conditions. Examples of constitutive plant promoters useful for expressing heterologous polypeptides in plant cells include, but are not limited to, the cauliflower mosaic virus (CaMV) 35S promoter, which confers constitutive, high-level expression in most plant tissues including monocots; the nopaline synthase promoter and the octopine synthase promoter. The expression cassette is usually provided in a DNA or RNA construct which is typically called an “expression vector” which is any genetic element, e.g., a plasmid, a chromosome, a virus, behaving either as an autonomous unit of polynucleotide replication within a cell (i.e. capable of replication under its own control) or being rendered capable of replication by insertion into a host cell chromosome, having attached to it another polynucleotide segment, so as to bring about the replication and/or expression of the attached segment. Suitable vectors include, but are not limited to, plasmids, bacteriophages, cosmids, plant viruses and artificial chromosomes. The expression cassette may be provided in a DNA construct which also has at least one replication system. In addition to the replication system, there will frequently be at least one marker present, which may be useful in one or more hosts, or different markers for individual hosts. The markers may a) code for protection against a biocide, such as antibiotics, toxins, heavy metals, certain sugars or the like; b) provide complementation, by imparting prototrophy to an auxotrophic host: or c) provide a visible phenotype through the production of a novel compound in the plant. Exemplary genes which may be employed include neomycin phosphotransferase (NPTII), hygromycin phosphotransferase (HPT), chloramphenicol acetyltransferase (CAT), nitrilase, and the gentamicin resistance gene. For plant host selection, non-limiting examples of suitable markers are β-glucuronidase, providing indigo production, luciferase, providing visible light production, Green Fluorescent Protein and variants thereof, NPTII, providing kanamycin resistance or G418 resistance, HPT, providing hygromycin resistance, and the mutated aroA gene, providing glyphosate resistance.

The term “promoter activity” refers to the extent of transcription of a polynucleotide sequence, homologue, variant or fragment thereof that is operably linked to the promoter whose promoter activity is being measured. The promoter activity may be measured directly by measuring the amount of RNA transcript produced, for example by Northern blot or indirectly by measuring the product coded for by the RNA transcript, such as when a reporter gene is linked to the promoter. The term “operably linked” refers to linkage of a DNA segment to another DNA segment in such a way as to allow the segments to function in their intended manners. A DNA sequence encoding a gene product is operably linked to a regulatory sequence when it is ligated to the regulatory sequence, such as, for example a promoter, in a manner which allows modulation of transcription of the DNA sequence, directly or indirectly. For example, a DNA sequence is operably linked to a promoter when it is ligated to the promoter downstream with respect to the transcription initiation site of the promoter and allows transcription elongation to proceed through the DNA sequence. A DNA for a signal sequence is operably linked to DNA coding for a polypeptide if it is expressed as a pre-protein that participates in the transport of the polypeptide. Linkage of DNA sequences to regulatory sequences is typically accomplished by ligation at suitable restriction sites or adapters or linkers inserted in lieu thereof using restriction endonucleases known to one of skill in the art.

The term “heterologous DNA” and or “heterologous RNA” refers to DNA or RNA that does not occur naturally as part of the genome or DNA or RNA sequence in which it is present, or that is found in a cell or location in the genome or DNA or RNA sequence that differs from that which is found in nature. Heterologous DNA and RNA (in contrast to homologous DNA and RNA) are not endogenous to the cell into which it is introduced, but has been obtained from another cell or synthetically or recombinantly produced. An example is a gene isolated from one plant species operably linked to a promoter isolated from another plant species. Generally, though not necessarily, such DNA encodes RNA and proteins that are not normally produced by the cell in which the DNA is transcribed or expressed. Similarly exogenous RNA encodes for proteins not normally expressed in the cell in which the exogenous RNA is present. Heterologous DNA or RNA may also refer to as foreign DNA or RNA. Any DNA or RNA that one of skill in the art would recognize as heterologous or foreign to the cell in which it is expressed is herein encompassed by the term heterologous DNA or heterologous RNA. Examples of heterologous DNA include, but are not limited to, DNA that encodes proteins, polypeptides, receptors, reporter genes, transcriptional and translational regulatory sequences, selectable or traceable marker proteins, such as a protein that confers drug resistance, RNA including mRNA and antisense RNA and ribozymes. In a particular embodiment also homologous DNA sequences derived from Veratrum californicum can be overexpressed or underexpressed in the isolated plant cell lines of the invention.

One approach that has been given interesting results for better production of plant secondary metabolites is elicitation. Elicitors are compounds capable of inducing defence responses in plants. These are usually not found in intact plants but their biosynthesis is induced after wounding. One of the commonly used elicitors are jasmonates, mainly jasmonic acid and its methyl ester, methyl jasmonate. Jasmonates are linoleic acid derivatives of the plasma membrane and display a wide distribution in the plant kingdom. Methyl jasmonate (MeJA) is known to induce the accumulation of numerous defence-related secondary metabolites (e.g. phenolics, alkaloids and sesquiterpenes) through the induction of genes coding for the enzymes involved in the biosynthesis of these compounds in plants. Jasmonates can modulate gene expression from the (post)transcriptional to the (post)translational level, both in a positive as in a negative way. Genes that are upregulated are e.g. defence and stress related genes (PR proteins and enzymes involved with the synthesis of phytoalexins and other secondary metabolites) whereas the activity of housekeeping proteins and genes involved with photosynthetic carbon assimilation are down-regulated. Cyclopamine and related compounds such as jervine can be measured by methods known in the art. Such methods comprise analysis by thin-layer chromatography, high pressure liquid chromatography, capillary chromatography, (gas chromatographic) mass spectrometric detection, radioimmuno-assay (RIA) and enzyme immuno-assay (ELISA).

The transformation of plant cell lines may be carried out by any suitable means, including by Agrobacterium-mediated transformation and non-Agrobacterium-mediated transformation, as discussed in detail below. Plants can be regenerated from the transformed cell (or cells) by techniques known to those skilled in the art. Where chimeric plants are produced by the process, plants in which all cells are transformed may be regenerated from chimeric plants having transformed germ cells, as is known in the art. Methods that can be used to transform plant cells or tissue with expression vectors of the present invention include both Agrobacterium and non-Agrobacterium vectors. Agrobacterium-mediated gene transfer exploits the natural ability of Agrobacterium tumefaciens to transfer DNA into plant chromosomes and is described in detail in Gheysen, G., Angenon, G. and Van Montagu, M. 1998. Agrobacterium-mediated plant transformation: a scientifically intriguing story with significant applications. In K. Lindsey (Ed.), Transgenic Plant Research. Harwood Academic Publishers, Amsterdam, pp. 1-33 and in Stafford, H. A. (2000) Botanical Review 66: 99-118. A second group of transformation methods is the non-Agrobacterium mediated transformation and these methods are known as direct gene transfer methods. An overview is brought by Barcelo, P. and Lazzeri, P. A. (1998) Direct gene transfer: chemical, electrical and physical methods. In K. Lindsey (Ed.), Transgenic Plant Research, Harwood Academic Publishers, Amsterdam, pp. 35-55. Hairy root cultures can be obtained by transformation with virulent strains of Agrobacterium rhizogenes. Protocols used for establishing of hairy root cultures vary, as well as the susceptibility of plant species to infection by Agrobacterium (Sevón N and Oksman-Caldentey K-M (2002) Planta Med. 68: 859-868; Vanhala L. et al. (1995) Plant Cell Rep. 14, 236). It is known that the Agrobacterium strain used for transformation has a great influence on root morphology and the degree of secondary metabolite accumulation in hairy root cultures. It is possible that by systematic clone selection e.g. via protoplasts, to find high yielding, stable, and from single cell derived-hairy root clones (Sevón N et al. (1998) Planta Med. 64: 37-41. This is possible because the hairy root cultures possess a great somaclonal variation. Another possibility of transformation is the use of viral vectors (Turpen T H (1999) Philos Trans R Soc Lond B Biol Sci 354(1383): 665-73).

The recombinant DNA and molecular cloning techniques applied in the below examples are all standard methods well known in the art and are e.g. described by Sambrook et al. (1989) Molecular cloning: A laboratory manual, second edition, Cold Spring Harbor Laboratory Press. Methods for tobacco cell culture and manipulation applied in the below examples are methods described in or derived from methods described in Nagata et al. (1992) Int. Rev. Cytol. 132, 1.

### EXAMPLES

### 1. Initiation and Maintenance of Callus Cultures from Veratrum californicum

Several aspects (e.g. influence of embryo size, culture medium and growth regulators) were studied in detail in order to clarify their role in the induction of embryogenesis. For monocotyledonous plant tissue culture it is known that embryo size, indicating the physiological stage of the embryo development, plays an important role in somatic embryogenesis and is critical for the establishment of embryogenic callus. The influence of embryo size of mature seeds on somatic embryogenesis of V. californicum was studied (Table 1). Embryo size significantly affected embryogenic callus induction (P<0.001). Bigger embryos (>2 mm) produced significantly higher embryogenic callus yield than smaller embryos (<2 mm). It was found that embryogenic callus induction from small embryos was difficult. Most probably small mature embryos had not developed well and had less active cells compared to big embryos. In previous works for immature embryo culture of monocot cereal plants, optimal embryo size was shown to vary from 0.5-2 mm (15), (16). Our results indicate that the influence of embryo size on somatic embryogenesis differs between mature and immature embryo culture, and therefore requirements for embryo sizes are different from the prior art. An interaction of embryo size and medium was not found (P>0.05). Culture medium, providing both nutrients and an osmotic environment, is an important factor influencing somatic embryogenesis. Composition of basal medium including the carbon source, the source and amount of total nitrogen, vitamins and growth regulators are crucial for embryogenic callus induction. Requirements for medium vary with species, genotype and culture conditions. For embryogenic callus induction of V. californicum, culture media and plant growth regulators were compared (Table 2). Both media (P<0.001) and plant growth regulators (P<0.001) significantly influenced embryogenesis. Among the five tested media mMS produced the highest embryogenic callus yield. Addition of picloram resulted in the highest embryogenic callus production. Interactions of media and growth regulators on embryogenesis were not found (P>0.05). In the present invention addition of amino acids (glutamine and casein hydrolysate) to MS significantly improved the embryogenic callus induction of V. californicum. AA medium, i.e. a medium enriched with glutamine and other amino acids as nitrogen source, was found not to be suitable for embryogenic callus induction (Table 2). Exogenous addition of growth regulators to culture medium is usually necessary for embryogenic callus induction. The type and concentration of auxin in induction medium plays an important role in obtaining high efficiency of somatic embryogenesis. Requirements vary with species, genotype and plant growth conditions (20). Effects of different concentrations of plant growth regulators in media were compared for V. californicum. In Table 3 it is shown that somatic embryogenesis was significantly influenced by the levels of growth regulators (P<0.001). Concentrations 4 mg/l and 7 mg/l gave significantly higher embryogenic efficiencies than 2 mg/l. Significant differences between concentrations of 4 mg/l and 7 mg/l were not found. Efficiency of auxins on somatic embryogenesis has been investigated previously for monocot cereal crops (15). The auxin 2,4-D has most often been used in induction medium for embryo culture initiation. Our results indicate that picloram is more suitable than 2,4-D, and is the best of the four growth regulators tested (Table 2). We could not find a significant (P>0.05) difference of embryogenic frequency between picloram and NAA suggesting that NAA would be another choice for embryogenic callus induction of V. californicum. Oxidative browning has been a major problem associated with plant tissue culture and limited the application of the culture techniques in many plants (24). Also a high percentage of V. californicum embryos released phenolic compounds during embryo culture. Culture medium (P<0.001) and growth regulators (P<0.001) significantly influenced phenolic exudation. The lowest percentage (18.9%) of embryos producing phenolic exudates was observed on mMS medium containing picloram. Browning is considered to be the result of oxidation of phenolic exudates released from plant cells into the surrounding medium and can even cause necrosis of plant cells. Supplementing the media with polyvinylpyrrolidone and activated charcol did not prevent browning of the Veratrum cultures.

### 2. Generation of Plant Suspension Cell Lines

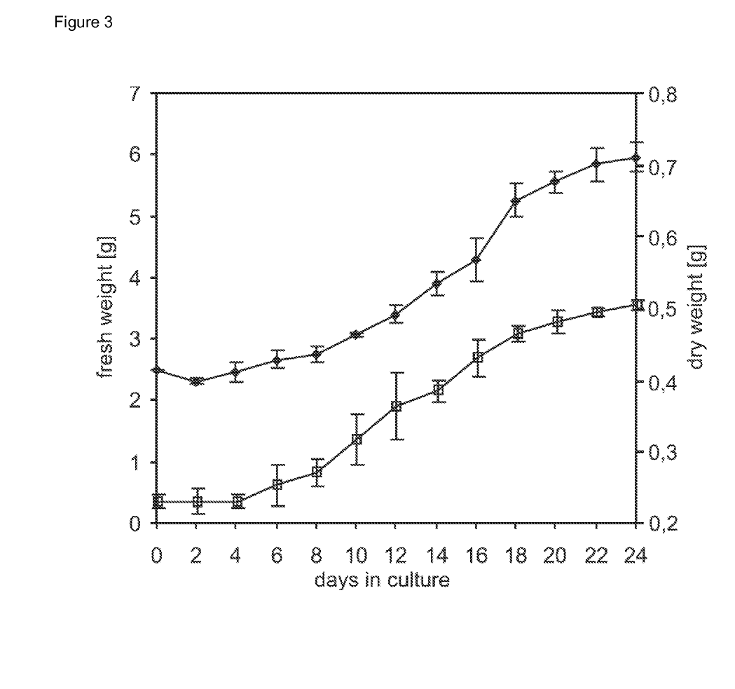

Embryogenic suspension cultures are finely dispersed and fast growing. Embryogenic cells aggregate in small groups and are highly cytoplasmic and non-vacuolated. The initiation of suspension cultures from isolated embryos of V. californicum resulted in A, B and C suspension lines grown in L2-medium. All these lines were also able to grow without growth regulators. In order to maintain the viability of the cultures, the cell suspensions were subcultured at the beginning of the stationary phase. According to earlier studies Lilium suspensions have been initiated from embryogenic calli, shoot apices and meristematic nodular cell clumps (21), (27). Liquid media MS, N6 or derivatives of them with different auxins have been commonly employed as culture media. AA medium, an amino acid based culture medium (14), has been used as culture medium for rapid establishment of rice suspension culture (29). This finding is in accordance with our observation on V. californicum. AA medium with the optimal concentration level of 4 mg/l NAA or 2,4-D improved the growth of the suspension line B. However, the long term maintenance of suspensions was better with NAA than with 2,4-D. The growth of suspension cell line B is shown in FIG. 3. Our result differs from previous work for monocotyledonous plants where the medium suitable for embryogenic callus induction has been also suitable for suspension culture.

### 3. Cryopreservation of Plant Suspension Cell Lines

The produced suspension lines A, B and C were cryopreserved by using a classical slow-freezing protocol. A summary of the cryopreserved lines is shown in Table 4. The lines were deposited in the VTT Culture Collection (VTT, Espoo, Finland). VTT Culture Collection codes will be used hereafter to specify the suspension lines. A recovery rate of 83% for the cryopreserved lines was recorded. For successful cryopreservation, the initiation of the dehydration procedure must be started when the suspensions are in the beginning of the exponential growth phase. The cells in exponential growth phase survive the freezing-thawing procedure better than the larger more vacuolized cells already reaching the stationary phase (30). This is most probably due to the nature of the cells in exponential growth phase: dense cytoplasm, small vacuoles and low water content. Osmotic dehydration was needed and 5% (v/v) DMSO was sufficient as a cryoprotectant. Dehydration has a positive influence on the freezing tolerance (31) since cell damage caused by high intracellular water content is prevented. In addition, the use of DMSO improves the viability and cell recovery as observed earlier (32). This may be due to the fact that the protection mechanism of DMSO is different when compared to sugar alcohols, since DMSO penetrates the cell membranes. The immediate thawing in a +40° C. water bath gave the best recovery for the cryopreserved lines. The cells have been kept under liquid nitrogen so far for one year, and been thawed successfully.

### 4. Regeneration of Green Plants from Embryogenic Calli and from Plant Suspension Cell Lines

Green plants of V. californicum were successfully regenerated from embryogenic calli maintained on solid medium. Regeneration abilities of the embryogenic calli sub-cultured on solid medium mMS (with 4 mg/l picloram) were evaluated on L2 medium (without growth regulators). The percentage of green plants regenerated from the embryogenic calli was excellent i.e. 107% (green plants/100 calli) after 3 months culture (FIG. 2). Maintenance of embryogenic capability and regeneration potential has been a critical problem in efficient in vitro culture systems (15). In the case of V. californicum regeneration capacity also decreased but only to 73% after 27 months culture. Whole green plant regeneration is crucial requirement for most current methods of plant tissue culture and genetic transformation. Rooted green plants were also successfully weaned in the green house. A fraction containing free steroid alkaloids was extracted from in vitro plantlets of V. californicum and subjected to liquid chromatography (LC) which was monitored by a mass spectrometer. The reference alkaloids cyclopamine, jervine, veratramine and solanidine were analysed to compare retention times and mass spectra. Veratramine and cyclopamine were detected in the plantlets. The identity of these compounds was confirmed by samples spiked with authentic references. Jervine has been reported from V. californicum earlier (25) and solanidine has been found in several Solanum and Veratrum species. Both veratramine and jervine are biosynthethic products of cyclopamine which is derived from cholesterol (26). Plant material collected from the wild can contain on average 0.35 g total alkaloids per 100 g dry material depending on the location and growth stage (27). The targeted LC method for the analysis of steroid alkaloids was chosen because it avoids a derivatisation step which is usually employed for alternative gas chromatography analysis. Low solubility and problems with bad peak shape were circumvented by the selected solvent mixture as described before (25). In addition, regeneration of green plants was established starting from plant suspension cell lines. Also upon elicitation with methyl-jasmonate these cell lines showed the production of cyclopamine and jervine.

### 5. Generation of Recombinant Plant Cell Lines

Recombinant cell lines of Veratrum californicum DURAND were produced by Agrobacterium tumefaciens and A. rhizogenes-mediated systems. In addition, direct gene transfer techniques such as particle bombardment and protoplast-based techniques were applied. The isolation and regeneration of protoplasts were utilized for the production of cell clones from the initial mixed populations.

### 6. Production of Cyclopamine in the Veratrum Plant Cell Lines

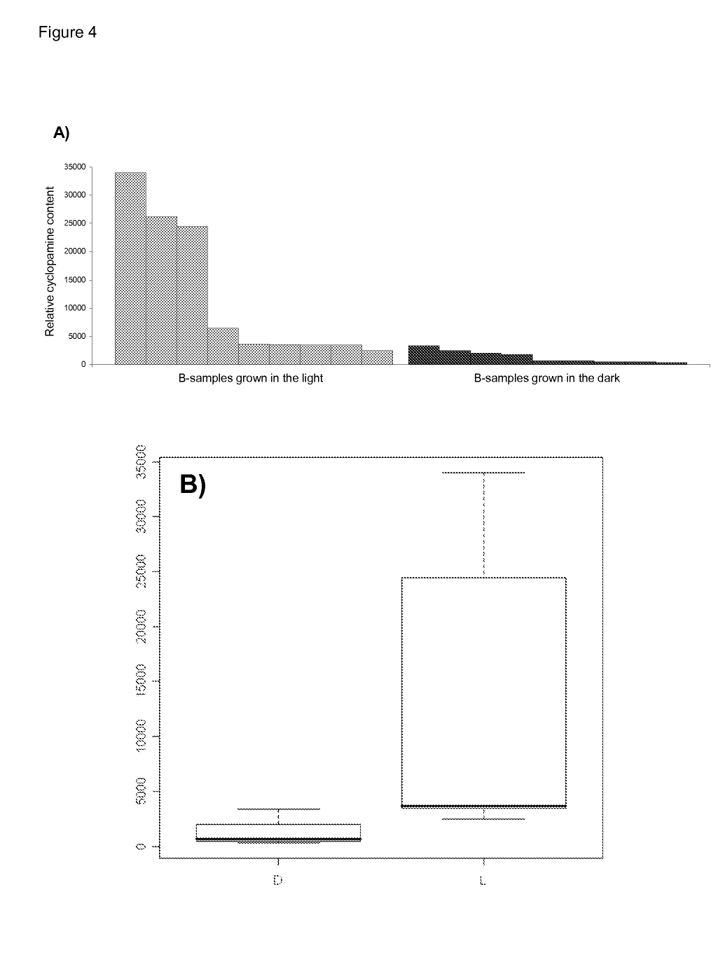

Cyclopamine and jervine could be detected in the plant cell lines. Since the B cell line grew faster than the A and C cell lines it was chosen for further analyzing the production of cyclopamine in response to the application of elicitors. The best elicitor proved to be light and indeed it was observed that the B-line grown in the light had a nine-fold increase in the cyclopamine content when compared to the same line grown in the dark conditions (see FIG. 4).

### Materials and Methods

### 1. Initiation and Maintenance of Callus Cultures

Seeds of Veratrum californicum Durand were peeled, rinsed with 94% ethanol and surface sterilized with 1% (v/v) sodium hypochlorite supplemented with a few drops of Tween20 for 10 minutes, and finally rinsed three times with sterile water. Seeds were allowed to germinate for 2 days at 22° C. in the dark on moist filter paper. Embryos were excised and cultured on MS medium (11) solidified with gelrite (3% w/v) and supplemented with 1 mg/l kinetin and 1 mg/l NAA in the dark at 22° C. Produced calli were subcultured to fresh plates in two to three weeks intervals. After four months of culture calli were transferred to L2-medium (12) solidified with gelrite (3% w/v) and supplemented with 2.5 mg/l 2,4-D and were subsequentially subcultured to fresh plates at one month intervals. Embryogenic callus cultures from isolated V. californicum embryos were induced by the following media: Basal media MS (11), R2M (13), L2 (12), modified MS medium (mMS, consisting of MS basic salts and vitamins, 146 mg/l glutamine, 200 mg/l casein hydrolysate) and AA (14) supplemented with different hormones (picloram, NAA, 2,4-D, dicamba) at three levels (2, 4 and 7 mg/l) all solidified with gelrite (3% w/v). The cultures were kept in the dark, at 25° C. and embryogenic calli were subcultured at one month intervals.

### 2. Plant Regeneration

In order to test the regeneration abilities of embryogenic calli during the subculturing, some of the calli were transferred to solid regeneration medium L2 (without hormones), and cultured in light (light intensity about 30-40 μmol mm⁻² s⁻¹ Osram cool white/Osram fluora, 1:1 on Watt basis) at 25° C. After 4-5 weeks green shoots were moved to hormone-free medium R2M in a plastic container (Greiner Bio-one 68/11 mm) for root development. Well developed plantlets were potted into peat soil and transferred to the greenhouse.

### 3. Extraction and Targeted HPLC/ESI/MS of Plantlets

100 mg lyophilized plant material was suspended in 4 ml 5% NH₄OH and extracted with 20 ml toluene for 10 min in an ultrasonic bath. Following centrifugation (7000 rpm, 10 min) the organic layer was collected and the sample residue was twice re-extracted with another 20 ml toluene. The organic phases were combined and the solvent was evaporated to dryness. For analysis the extract was redissolved in 200 μl of the LC-solvent which is described below. HPLC separation was performed using a Waters HT-Alliance 2795 system and was monitored with a Micromass Quattro Micro triple quadruple mass spectrometer equipped with an electrospray source. The ion source was operated at capillary voltage 4.00 kV and cone voltage 60 V. Source and desolvation temperatures were 130° C. and 290° C., respectively. Desolvation gas flow was 911 l/h and cone gas flow 30 l/h. The scan mode function was applied to record the protonated molecular ions (m/z 200-900). An aliquot of 50 μl of sample was loaded onto a reverse-phase C18 column (Xterra MS C18, 4.6×150 mm, 5 μm, Waters) at 35° C. The sample was eluted within 30 min using isocratic conditions of acetonitrile and 0.1% TFA (30:70) applying a flow of 1 ml/min and a split of 0.2 ml/min reaching the mass spectrometer. Commercially available alkaloids cyclopamine, jervine, veratramine and solanidine were used as reference compounds.

### 4. Initiation of Cell Suspension Cultures

Suspensions were initiated from Veratrum californicum calli grown on solid L2-medium. They were maintained in liquid L2-medium with 2,4-D (2.5 mg/l) and without hormones. Suspensions were subcultured in 10 to 14 days intervals by subculturing 20 ml of the 10 days old suspension (2-3 g fresh weight cells) to 30 ml of fresh medium. Produced suspension lines A, B and C were grown at +25° C. on a rotary shaker (90 rpm) in the dark.

### 5. Cryopreservation of the Cell Suspension Lines A, B and C

The suspension lines A, B and C were cryopreserved by using a Kryo10 device (Planer Biomed). The suspensions were subcultured 4 to 5 days prior to the start of the cryopreservation experiment. A dehydration procedure as follows was applied: I) Sugar alcohol concentration of the suspension was adjusted to 0.2 M by adding five times small aliquots of 4 M sorbitol during a period of 30 minutes. After sorbitol additions, the suspensions were incubated under normal growth conditions for 24 hours (in the dark, 25° C., on a rotary shaker (90 rpm)). II) Sugar alcohol concentration was adjusted to 0.4 M by 4 M sorbitol and incubations were carried out as in step I). The dehydrated suspensions were transferred to ice and DMSO was added to reach 5% (v/v) concentration. Cell:medium ratio was adjusted to 1:2 and suspensions were packed into ampulles. Protectant-treated suspensions were kept on ice for a period of 100 minutes. A freezing protocol as follows was applied: I) A rate of 10° C./min to reach 0° C. was followed by II) incubation at 0° C. for 20 minutes. The freezing was finalized by using III) a rate of 1° C./min to reach −40° C. and then IV) samples were immersed in liquid nitrogen. Thawing of cryopreserved samples was carried out by immersing the suspension ampulles straight from liquid nitrogen to a 40° C. water bath for 2 minutes. Cells were transferred to sterile filter paper on solid culture medium originally used for the culture of that particular cell suspension line. Cell division and growth of the cultures were monitored.

### 6. Growth Optimization of the Suspension Cell Line B

Growth of suspension line B on L2-medium with 2,4-D (2.5 mg/l) was further optimized. About 3 g (fw) of cells were transferred to an Erlenmeyer flask containing 50 ml liquid AA medium (with 2,4-D and NAA at levels of 2, 4 and 7 mg/l or without hormones), and incubated in the light (light intensity about 30-40 μmol mm⁻² s⁻¹ Osram cool white/Osram fluora, 1:1 on watt basis) on a rotary shaker (130 rpm) at 25° C. The volume of the liquid medium was added up to 100 ml after the second subculture. The suspensions were sub-cultured at three weeks intervals by replacing about half of the old culture with an equal volume of fresh medium.

### 7. Statistical Analysis

Frequencies of embryos producing embryogenic calli and frequencies of regenerated green and albino plants were recorded. Oxidative browning percentages of embryos were calculated. Completely randomized designs (CRD) were used in the experiments. Each Petri-dish with 20 embryos was considered an experimental unit and each treatment contained five replicates. Data analyses with two treatment levels were carried out by t-test, and with more than two treatment levels by the ANOVA procedure. Multi-range comparisons were performed by the LSD test.

### 8. Transformation Protocols

8.1 Agrobacterium-Mediated Transformation of Veratrum californicum DURAND

Agrobacterium tumefaciens (LBA4404, carrying the gene of interest and a selection marker gene nptII both under 35S-promoter) was grown for two nights at +28° C. in 5 ml of LB supplemented with 20 ppm rifampicin, 20 ppm gentamycin, 20 ppm streptomycin and 100 ppm spectinomycin. Approximately 3 to 5 g (fw) of Veratrum californicum suspension cells or calli were immersed to 3 to 5 ml of Agrobacterium suspension in a Petri dish with slight shaking. After 10 to 20 minutes of infection the bacteria was removed by pipetting and the Veratrum cells were blotted on sterile filter paper to remove the extra bacteria. The Veratrum cells were transferred to solid L2-medium plate (without 2,4-D, supplemented with 100 or 200 μM acetosyringone. L2-medium is described by Lazzeri et al. 1991) and co-cultivated in the dark at room temperature for 3 to 5 days. After co-cultivation period the infected Veratrum cells were transferred to a selection plate (L2 without 2,4-D, supplemented with 500 ppm vancomycin, 500 ppm carbenisillin and 50 ppm of kanamycin or 30 ppm of geneticin).

8.2 Production of Transgenic Cell Lines of Veratrum californicum DURAND by Particle Bombardment

Approximately 200 mg (fw) of calli or suspension culture was place on solid L2-medium. The expression construct contained the gene of interest and a selection marker gene nptII, both under control of 35S-promoter. Particle bombardment by PDS/1000-He (BioRad) was carried out according to manufacturer's instructions. The bombarded samples were allowed to recover over night at 25° C. in the dark. The day after bombardment, the Veratrum cells were transferred to a selection plate (L2 without 2,4-D, supplemented with 50 ppm of kanamycin or 30 ppm of geneticin).

8.3 Isolation and Culture of Veratrum californicum DURAND Protoplasts

Two to four week old Veratrum calli and suspension mass harvested 3 to 5 days after subculture were used for protoplast isolation. Approximately 1.0 g (fw) of calli or 0.5 g (fw) of suspension cell mass was incubated in 15 ml of enzyme solution containing 1.0% (w/v) cellulase Onozuka RS, 0.5% (w/v) Macerozyme R10 and 0.05% (w/v) pectolyase Y23 in LW-solution (Lazzeri et al. 1991). After 3-4 hour incubation the suspension was filtered through 100 μm and 48 μm nylon sieves. The protoplasts were washed with LW solution. The isolated protoplast were purified with a 20% (w/v) maltose gradient (100 g, 5 min centrifugation).

L1-medium (Lazzeri et al. 1991) was used for the protoplast culture. The medium was supplemented with 0.5M maltose and 1.2% (w/v) agarose (SeaPlaque™). The protoplasts were plated at density of 1-2 million protoplast per sample on Millicell-CM culture plate inserts. The inserts were placed in 5 cm Petri dishes with 7 ml of nurse culture. The initial cell line grown in L2 medium used for protoplast isolation was used as a nurse culture. Protoplast cultures were incubated on a rotary shaker (65 rpm, stroke radius 2.5 cm) at 25° C. in the dark. After one week of culture, the nurse culture was removed and replaced by 7 ml of fresh L2 medium. After 4 to 6 weeks of culture regenerating microcalli were picked to a solid L2-medium.

8.4 Production of Transgenic Cell Lines of Veratrum californicum DURAND via Protoplast-Based Techniques as PEG and Electroporation

For electroporation, the isolated and purified protoplasts were placed to electroporation buffer (0.55M mannitol, 35 mM aspartic acid monopotassium salt, 35 mM, glutamic acid monopotassium salt, 5 mM calcium gluconate, 5 mM MES, pH 7.0) at the density of 2-6 million protoplasts/ml. For electroporation protoplast aliquots of 300 μl were mixed with 30 μg of plasmid DNA carrying the gene of interest and a selection marker both under control of 35S promoter. The samples were chilled on ice 10 min before applying an electrical field of 800 V/cm by discharge of a 200 μF capacitor. The protoplast were kept on ice for 10 min, after which they were plated for culturing as describe above in Isolation and culture of Veratrum californicum DURAND protoplasts. After 4 to 6 weeks of culture regenerating microcalli were picked to a solid L2-medium without selective agent or supplemented with 30-70 ppm of geneticin.

For the polyethylene glycol-mediated transformation, isolated and purified protoplasts were resuspended to a following buffer: 140 mM NaCl, 5 mM KCl, 5 mM HEPES, 5 mM glucose, 125 mM CaCl₂, pH 7.0 at the density of 2-6 million protoplasts/ml. Protoplast aliquots of 500 μl were mixed with 50 μg of plasmid DNA carrying the gene of interest and a selection marker both under control of 35S promoter. Up to 1 ml of PEG solution (PEG 4000 (Fluka), 40% v/v in above buffer) was added dropwise with gentle shaking. The mixture was incubated for 15 ml with gentle shaking intervals. The buffer was added in aliquots of 2 ml in 5 min intervals for four times. The PEG-treated protoplast were centrifuged (100 g, 5 min) and plated for culturing as describe above in Isolation and culture of Veratrum californicum DURAND protoplasts. After 4 to 6 weeks of culture regenerating microcalli were picked to a solid L2-medium without selective agent or supplemented with 30-70 ppm of geneticin.

Composition of L2-medium (without 2,4-D), LW-washing solution and L1-medium (Lazzeri, P. A. et al. (1991) Theor. Appl. Genet. 81:437-444)

| | L2 | LW | L1 |
|---|---|---|---|
| COMPOSITION: | mg/l | mg/l | mg/l |
| NH₄NO₃ | 1500 | 750 | 750 |
| KH₂PO₄ | 200 | 200 | 200 |
| KNO₃ | 1750 | 1750 | 1750 |
| CaCl₂•2H₂O | 450 | 450 | 450 |
| MgSO₄•7H₂O | 350 | 350 | 350 |
| FeSO₄•7H₂O | 27.8 | — | 27.8 |
| Na₂EDTA•2H₂O | 37.3 | — | 37.3 |
| MnSO₄•H₂O | 15 | 15 | 15 |
| H₃BO₃ | 5 | 5 | 5 |
| ZnSO₄•7H₂O | 13.35 | 13.35 | 13.35 |
| KI | 0.75 | 0.75 | 0.75 |
| NaMoO₄•2H₂O | 0.27 | 0.27 | 0.27 |
| CuSO₄•5H₂O | 0.025 | 0.025 | 0.025 |
| CoCl₂•6H₂O | 0.025 | 0.025 | 0.025 |
| Nicotinic acid | 1 | — | 1 |
| Thiamine-HCl | 10 | — | 10 |
| Pyridoxine-HCl | 1 | — | 1 |
| Ascorbic acid | 1 | — | 1 |
| Ca-panthothenate | 1 | — | 0.5 |
| Choline chloride | — | — | 0.5 |
| Folic acid | — | — | 0.2 |
| p-Aminobenzoic acid | — | — | 0.01 |
| Biotin | — | — | 0.005 |
| Vitamin A | — | — | 0.005 |
| Vitamin D₃ | — | — | 0.005 |
| Glutamine | 750 | 750 | 750 |
| Proline | 150 | 150 | 150 |
| Aspargine | 100 | 100 | 100 |
| m-inositol | 200 | — | 100 |
| Maltose | 30 000 | — | 180 000 |
| Mannitol | — | 110 000 | 125 |
| Sucrose | — | — | 125 |
| Mannose | — | — | 125 |
| Fructose | — | — | 125 |
| Ribose | — | — | 125 |
| Xylose | — | — | 125 |
| Rhamnose | — | — | 125 |
| Cellobiose | — | — | 125 |
| Sorbitol | — | — | 125 |
| Coconut water (ml/l) | — | — | 10 |

### 9. Analysis of Cyclopamine and Jervine Produced by Veratrum Plant Cell Lines

100 μl 0.5% SDS solution and 50 μl 25% NH4OH was added to 100 mg FW frozen cell powder. After mixing 1 ml toluene was added and the solution was extracted for 10 min in an ultrasonic bath. Following centrifugation the organic layer was collected and the sample residue was twice re-extracted with another 1 ml toluene. The organic phases were combined and the solvent was evaporated to dryness. The residue was then quantitatively transferred to a GC vial and re-dissolved in 80 μl CH2Cl2. An aliquot was directly injected into the GC.

GC separation was performed using an Agilent 6890 system and was monitored with an Agilent 5973 Network MS Quadropole. The selected ion mode function was applied to record the characteristic molecular ions (cyclopamine: 124, 396; jervine: 110, 124). Within a set of samples the response values for the specific ions were used to compare relative levels of alkaloids. A mixture of commercially available reference compounds (cyclopamine and jervine) was routinely analyzed for comparison of retention times and fragmentation.

TABLE 1
Influence of embryo size on embryogenic callus induction
(% of embryogenesis, data from 5 replicates)

| | Embryo size | | |
|---|---|---|---|
| Medium | <2 mm | 2 mm-4 mm | >4 mm |
| mMS | 26.58 | 82.57 | 84.32 |
| R2M | 25.05 | 77.48 | 76.33 |
| AA | 17.83 | 64.43 | 65.77 |
| Average | 23.15B | 74.82A | 75.48A |

LSD_{0.05} = 5.67 between embryo sizes A, B (P < 0.001)

TABLE 2
Effects of media and growth regulators on embryogenesis (% of embryogenesis,
data from 5 replicates)

| | Medium | | | | | |
|---|---|---|---|---|---|---|
| Growth regulators | mMS | R2M | 502 | MS | AA | Average |
| Picloram | 81.33 | 75.40 | 68.32 | 69.32 | 59.75 | 70.82a |
| NAA | 74.93 | 73.95 | 64.98 | 62.62 | 62.04 | 67.70a |
| Dicamba | 60.79 | 58.67 | 55.61 | 50.90 | 51.24 | 55.44bc |
| 2,4-D | 55.32 | 58.85 | 53.41 | 50.52 | 43.43 | 52.33c |
| Average | 68.12A | 66.72AB | 60.58BC | 58.34CD | 54.11D | |

LSD_{0.05} = 6.16 between media A, B, C, D (P < 0.001)
LSD_{0.05} = 5.51 between hormones a, b, c (P < 0.001)

TABLE 3
Influence of growth regulator concentrations in induction media on
embryogenesis (% of embryogenesis, data from 5 replicates)

| | Concentration of growth regulator | | | | | |
|---|---|---|---|---|---|---|
| | Picloram (mg/l) | | | NAA (mg/l) | | |
| Medium | 2 | 4 | 7 | 2 | 4 | 7 |
| mMS | 69.76 | 80.57 | 81.46 | 65.35 | 73.67 | 75.37 |
| R2M | 68.53 | 75.73 | 76.07 | 63.75 | 74.32 | 73.01 |
| AA | 51.43 | 62.59 | 59.97 | 48.95 | 64.21 | 68.06 |
| Average | 63.24B | 72.96A | 72.50A | 59.35b | 70.74a | 72.15a |

LSD_{0.05} = 6.08 between concentrations within Picloram (A, B) (P < 0.001)
LSD_{0.05} = 5.77 between concentrations within NAA (a, b) (P < 0.001)

TABLE 4
Cryopreserved Veratrum californicum suspension
lines and their VTT culture collection codes

| | Original | | |
|---|---|---|---|
| Collection code | cell line | Culture medium | Culture conditions |
| VTT-G-06005 | B | L2 with 2,4-D | 90 rpm, in the dark |
| VTT-G-06006 | B | L2 without 2,4-D | 90 rpm, in the dark |
| VTT-G-06007 | C | L2 with 2,4-D | 90 rpm, in the dark |
| VTT-G-06008 | C | L2 without 2,4-D | 90 rpm, in the dark |
| VTT-G-06009 | A | L2 with 2,4-D | 90 rpm, in the dark |
| VTT-G-06010 | A | L2 without 2,4-D | 90 rpm, in the dark |
| VTT-G-07011 | B | L2 without 2,4-D | 120 rpm, in the light |
| VTT-G-06012 | C | L2 without 2,4-D | 120 rpm, in the light |

### REFERENCES

¹Zomlefer W B, Whitten W M, Williams N H, Judd W S. An overview of Veratrum s.1. (Liliales: Melanthiaceae) and an infrageneric phylogeny based on ITS sequence data. Systematic Bot 2003; 28: 250-269
²James L F, Panter K E, Gaffield W, Molyneux R J. Biomedical applications of poisonous plant research. J Agric Food Chem 2004; 52: 3211-3230
³Cooper M K, Porter J A, Young K E, Beachy P A. Teratogen-mediated inhibition of target tissue response to Shh signaling. Science 1998; 280: 1603-1607
⁴Taipale J, Chen J K, Cooper M K, Wang B L, Mann R K, Milenkovic L et al. Effects of oncogenic mutations in Smoothened and Patched can be reversed by cyclopamine. Nature 2000; 406: 1005-1009
⁵Berman D M, Karhadkar S S, Hallahan A R, Pritchard J I, Eberhart C G, Watkins D N, et al. Medulloblastoma growth inhibition by Hedgehog pathway blockade. Science 2002; 297: 1559-1561
⁶Taipale J, Beachy P A. The Hedgehog and Wnt signaling pathways in cancer. Nature 2001; 411: 349-354
⁷Watkins D N, Berman D M, Burkholder S G, Wang B L, Beachy P A, Baylin S B. Hedgehog signalling within airway epithelial progenitors and in small-cell lung cancer. Nature 2003; 422: 313-317
⁸Goossens A, Häkkinen S T, Laakso I, Seppänen-Laakso T, Biondi S, De Sutter V et al. A functional genomics approach toward the understanding of secondary metabolism in plant cells. Proc Nat Acad Sci USA 2003; 100: 8595-8600
⁹Rischer H, Ore\check{s}i\check{c} M, Seppanen-Laakso T, Katajama M, Lammertyn F, Ardiles-Diaz W et al. Gene-to-metabolite networks for terpenoid indole alkaloid biosynthesis in Catharanthus roseus cells. Proc Nat Acad Sci USA 2006; 103: 5614-5619
¹⁰Oksman-Caldentey K M, Inzé D. Plant cell factories in the post-genomic era: new ways to produce designer secondary metabolites. Trends Plant Sci 2004; 9: 433-440
¹¹Murashige T, Skoog F. A revised medium for rapid growth and bioassay with tobacco tissue cultures. Physiol Plant 1962; 15: 473-497
¹²Lazzeri P A, Brettschneider R, Luhrs R, Lörz H. Stable transformation of barley via PEG-induced direct DNA uptake into protoplasts. Theor Appl Gen 1991; 81: 437-444
¹³Wang X, Hu H. The effect of potato II medium for triticale anther culture. Plant Sci Lett 1984; 36: 237-239
¹⁴Müller AG, Grafe R. Isolation and characterization of cell lines of Nicotiana tabacum lacking nitrate reductase. Mol Gen Genet: 1978; 161: 67-76
¹⁵Lührs R, Lörz H. Plant regeneration in vitro from embryogenesis cultures of spring- and winter-type barley (Hordeum vulgare L.) varieties. Theor Appl Gen 1987; 75: 16-25
¹⁶Ma R, Pulli S. Factors influencing somatic embryogenesis and regeneration ability in somatic tissue culture of spring and winter rye. Agricultural and Food Science 2004; 13: 363-377
¹⁷Nhut D T. Micropropagation of lily (Lilium longiflorum) via in vitro stem node and pseudo-bulblet culture. Plant Cell Rep 1998; 17: 913-916
¹⁸Chaudhuri D, Sen S. In vitro response of Scilla siberica. Scientia Horticulturae 2002; 95: 51-62
¹⁹Armstrong C L, Green C E. Establishment and maintenance of friable, embryogenic maize callus and the involvement of L-proline. Planta 1985; 164: 207-214
²⁰Sopory S K, Munshi M. Anther culture. In: Jain M S, Sopory S K, Veilleux R E, editors. Kluwer Academic Publishers, Dordrecht: 1996. p. 145-176
²¹Tribulato A, Remotti P C, Loffler H J M, van Tuyl J M. Somatic embryogenesis and plant regeneration in Lilium longiflorum Thunb. Plant Cell Rep 1997; 17: 113-118
²²Mori S, Adachi Y, Horimoto S, Suzuki S, Nakano M. Callus formation and plant regeneration in various Lilium species and cultivars. In Vitro Cellular & Developmental Biology-Plant 2005; 41: 783-788
²³Barro F, Martin A, Lazzeri P A, Barcelo P. Medium optimization for efficient somatic embryogenesis and plant regeneration from immature inflorescences and immature scutella of elite cultivars of wheat, barley and tritordeum. Euphytica 1999; 108: 161-167
²⁴Anthony J M, Senaratna T, Dixon K W, Sivasithamparam K. The role of antioxidants for initiation of somatic embryos with Conostephium pendulum (Ericaceae) Plant Cell Tissue Organ Cult 2004; 78: 247-252
²⁵Browne C A, Sim F R, Rae I D, Keeler R F. Isolation of teratogenic alkaloids by reversed-phase high-performance liquid chromatography. J Chromatogr 1984; 336:
²⁶Keeler R F, Binns W. Teratogenic compounds of Veratrum californicum as a function of plant part, stage and site of growth. Phytochemistry 1971; 10: 1765-1769
²⁷Kaneko K, Mitsuhashi H, Hirayama K, Ohmori S. 11-Deoxojervine as a precursor for jervine biosynthesis in Veratrum grandiflorum. Phytochemistry 1970; 9: 2497-2501
²⁸Horita M, Morohashi H, Komai F. Regeneration of flowering plants from difficile lily protoplasts by means of a nurse culture. Planta 2002; 215: 880-884
²⁹Jenes B, Pauk J. Plant regeneration from protoplast derived calli in rice (Oryza sativa L.) using dicamba. Plant Sci 1989; 63: 187-198
³⁰Menges M, Murray J A H. Cryopreservation of transformed and wild-type Arabidopsis and tobacco cell suspension cultures. Plant J 2004; 37: 635-644
³¹Smith A U, Ploge C, Smiles J. Microscopic observations of living cells during freezing and thawing. J R Microsc Soc 1951; 71: 186-195
³²Winkelmann T, Mussmann V, Serek M. Cryopreservation of embryogenic suspension cultures of Cyclamen persicum Mill. Plant Cell Rep 2004; 23: 1-8

1. An isolated plant cell line from Veratrum californicum.

2. The plant cell line according to claim 1 that produces jervine and/or cyclopamine.

3. A process for producing the plant cell line of claim 1, the process comprising:
a) isolating mature embryos from Veratrum californicum wherein said mature embryos are larger than 2 mm,
b) cultivating said isolated mature embryos on modified Murashige and Skoog salts medium supplemented with 4-7 mg/l picloram towards embryogenic callus, and
c) forming a plant suspension culture from said callus in an amino acid based medium supplemented with 4 mg/l 1-naphthaleneacetic acid.

4. The plant cell line according of claim 1, which is a recombinant plant cell line.

5. A method of producing jervine and/or cyclopamine, the method comprising:
utilizing the plant cell line of claim 1 to produce jervine and/or cyclopamine.

6. An in vitro shoot culture of Veratrum californicum.

7. A method of producing shoot cultures of Veratrum californicum, the method comprising:
utilizing the plant cell line of claim 1 to produce shoot cultures of Veratrum californicum.

8. The process of claim 3, wherein the plant cell line produces jervine and/or cyclopamine.

9. The method according to claim 5, wherein the plant cell line produces jervine and/or cyclopamine.

10. The method according to claim 7, wherein the plant cell line produces jervine and/or cyclopamine.
